(19) United States
(12) Patent Application Publication
(10) Pub. No.: US 2008/0091251 A1
(43) Pub. Date: Apr. 17, 2008

(54) METHODS AND APPARATUS FOR PROVIDING A SKIN TREATMENT DEVICE ON A PAY-PER-USE BASIS

(75) Inventors: Charles T. Walneck, Cary, IL (US); Christopher T. Walneck, Cary, IL (US); James H. Santee, Cary, IL (US); James W. Raim, Chicago, IL (US)

Correspondence Address: BELL, BOYD & LLOYD, LLP, P.O. Box 1135, CHICAGO, IL, 60690, US

(73) Assignee: SKINCARE TECHNOLOGY, INC., Chicago, IL (US)

(21) Appl. No.: 11/689,976

(22) Filed: Mar. 22, 2007

Related U.S. Application Data
(60) Provisional application No. 60/785,057, filed on Mar. 23, 2006.

(51) Int. Cl. A61N 5/06

(52) U.S. Cl. 607/91; 235/382; 606/9; 705/400

(57) ABSTRACT

Methods and apparatus for providing a skin treatment device on a pay-per-use basis are disclosed. Generally, a wholesaler supplies an LED skin treatment device to a retailer. The retailer pays the wholesaler on a per use basis, which is regulated by a digital key, such as a smart card attached to the treatment device via a smart card reader. When the value of the digital key drops below a threshold (e.g., only ten treatment sessions remaining), an alert is generated by the treatment device. When the value of the digital key is reduced to zero, the treatment device does not operate until the digital key is replenished or replaced.

### RELATED APPLICATIONS

This application claims the benefit of U.S. Provisional Patent Application No. 60/785,057, filed Mar. 23, 2006 entitled “Methods and Apparatus for Treating Hands with Light Emitting Diodes” the contents of which are hereby incorporated by reference.

### TECHNICAL FIELD

The present disclosure relates in general to skin treatment devices, and, in particular, to methods and apparatus for providing a skin treatment device on a pay-per-use basis.

### BACKGROUND

As people age, they tend to become more and more concerned with the appearance of their skin. As a result, these people try various remedies from lotions to surgery. Another method of reducing the appearance of aging is light treatments. Typically, a light based skin treatment device uses a plurality of lasers and/or light emitting diodes (LEDs) at specific frequencies. Such a skin treatment device typically delivers a carefully timed sequence of light frequencies to a user's skin.

However, existing skin treatment devices have certain drawbacks. Some skin treatment devices only include a few small light sources. For example, a small cluster of LEDs may be arranged in a two inch circle at the end of a treatment wand. As a result, the device must be moved from one skin location to another skin location to cover a significant area of skin. This greatly extends the overall treatment time and is a deterrent to many users. For example, if a user is attempting to treat all of the skin on both sides of both of his/her hands with a single small device, up to ten sessions of ten minutes each (e.g., 100 minutes) may be required for one application.

Other skin treatment devices include a large panel of light sources capable of covering a large area of skin. However, these skin treatment devices are still not capable of treating two different sides of an area simultaneously (e.g., both sides of a hand) . In addition, these skin treatment devices are relatively expensive. As a result, retailers (e.g., nail saloons and spas) are often not willing to take on the financial risk of purchasing one of these skin treatment devices. This risk aversion is especially acute in retailers who are new to light based skin treatment.

### SUMMARY

The system disclosed herein solves these problems. To reduce the duration of treatments, a partially enclosed system large enough for both hands of a typical user includes a lower panel of LEDs and an upper panel of LEDs. In this manner, both sides of both hands of a user may be treated in one session (e.g., ten minutes).

To reduce the financial risk to the retailer, the system is provided to retailers on pay-per-use basis (e.g., $1 per minute). If the retailer is able to sell a session (e.g., at $2 per minute), a profit is essentially guaranteed. If the retailer is not able to sell a significant number of sessions, the only down side is some sales effort and some retail space.

To account for the pay-per-use method, the system is regulated by a digital key. For example, a smart card or debit card may be attached to the treatment device via a card reader. When the value of the digital key drops below a certain threshold (e.g., only ten treatment sessions remaining), an alert is generated by the treatment device. When the value of the digital key is reduced to zero, the treatment device does not operate until the digital key is replenished or replaced.

### BRIEF DESCRIPTION OF THE DRAWINGS

FIG. 1 is an illustration of an example LED skin treatment device for hands.

FIG. 2 is a symbolic representation of an example skin treatment device with a lower illumination surface and an upper illumination surface.

FIG. 3 is a block diagram of the electrical systems of an example skin treatment device.

FIG. 4 is a block diagram of an example network environment suitable for replenishing payment cards.

FIG. 5 is a block diagram of the electrical systems of an example computing device.

FIG. 6 is a flowchart of an example process for paying for a skin treatment device on a per-use basis.

### DETAILED DESCRIPTION OF EXEMPLARY EMBODIMENTS

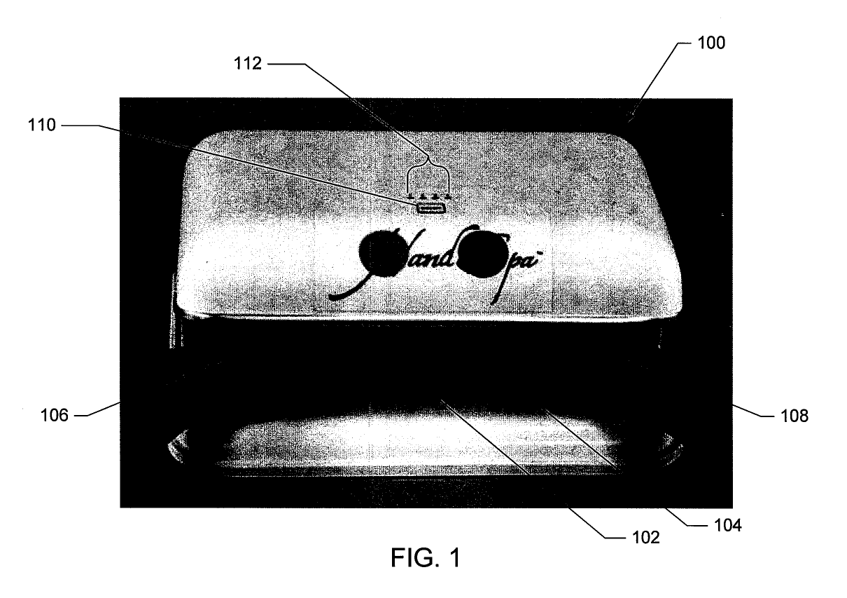

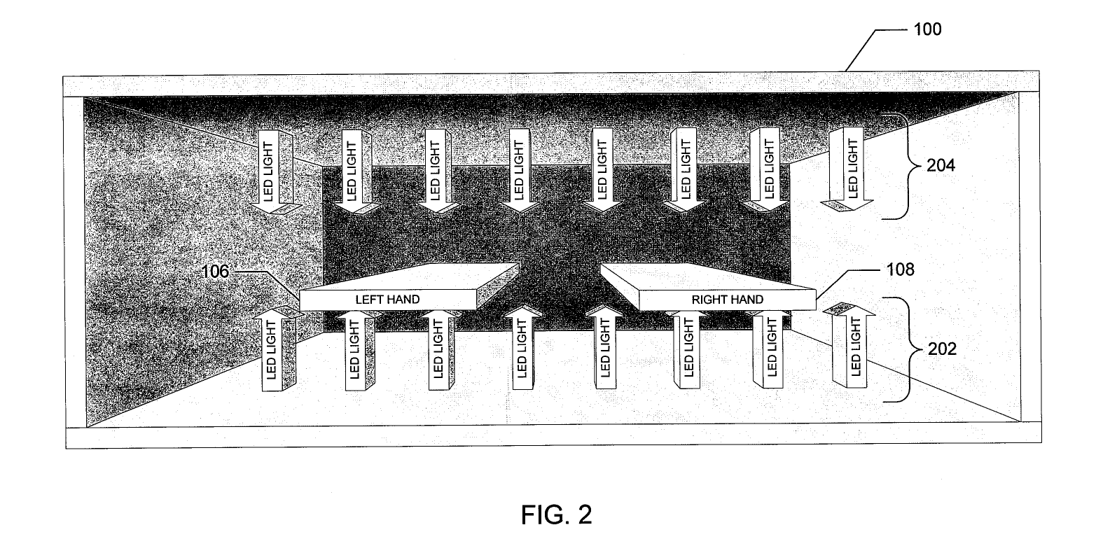

An illustration of an example LED skin treatment device 100 for hands is illustrated in FIG. 1. In this example, the LED skin treatment device 100 is a table top unit that includes a lower illumination surface 102 and an upper illumination surface 104. A symbolic representation of an example skin treatment device 100 with a lower illumination surface 102 and an upper illumination surface 104 is illustrated in FIG. 2. In some embodiments, the treatment device 100 only includes a lower illumination surface 102 or an upper illumination surface 104. A user preferably places his/her left hand 106 and his/her right hand 108 into the treatment device 100 simultaneously via an aperture in the front of the treatment device 100 as shown. However, in some embodiments, the user may place one hand at a time into the treatment device 100. A technician or the user then presses a start button 110 to begin a preprogrammed illumination session.

Each illumination session applies specific frequencies of visible and/or non-visible light to the user's skin. For example, the treatment device 100 may include a combination of visible red, visible amber, and non-visible infrared LEDs. In a preferred embodiment, the treatment device 100 includes six high intensity red emitters with diffusion optics. The six high intensity red emitters preferably have a primary frequency of 625 nanometers and a power level of 160 milliwatts. The preferred treatment device 100 also includes two high intensity amber emitters with diffusion optics. The two high intensity amber emitters preferably have a primary frequency of 590 nanometers and a power level of 80 milliwatts. The preferred treatment device 100 also includes 64 infrared LEDs, wherein each infrared LED has a primary frequency of 940 nanometers and a power level of 18 milliwatts. In such an embodiment, the effective photo power at 30 millimeters above the unit base is 2 mW/cm².

In addition, some or all of the light sources (e.g. LEDs) may be blinked (i.e., photo-pulsated) in a specific pattern and or frequency. For example, the light sources may be modulated at a frequency of 73 Hertz and 584 Hertz in a 80%/20% weighted application mix. The light sources shine onto the user's skin. However, the light sources preferably do not contact the user's skin.

When the illumination session begins, four status LEDs 112 light to indicate that 75%-100% of the session time remains. For example, four illuminated status LEDs 112 may indicate that approximately 7.5-10 minutes remain in the session. When the remaining session time falls below the 75% point (e.g., <7.5 minutes remaining), one of the four status LEDs 112 preferably turns off to indicate that 50%-75% of the session time remains. When the remaining session time falls below the 50% point (e.g., <5 minutes remaining), another one of the four status LEDs 112 preferably turns off (i.e., two LEDs on and two LEDs off) to indicate that 25%-50% of the session time remains. When the remaining session time falls below the 25% point (e.g., <2.5 minutes remaining), yet another one of the four status LEDs 112 preferably turns off (i.e., one LED on and three LEDs off) to indicate that 0%-25% of the session time remains. When the session ends, the last of the four status LEDs 112 preferably turns off to indicate that the session is complete. In addition, a transducer or other audio device may beep to indicate one or more of these transitions.

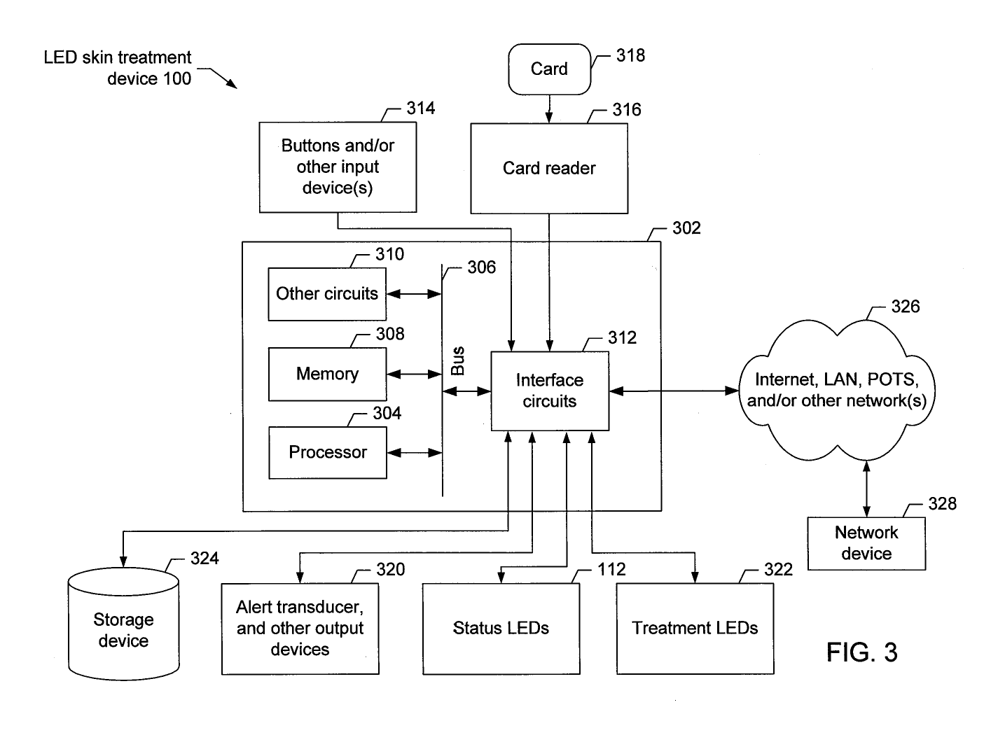

A block diagram of an example skin treatment device 100 is illustrated in FIG. 3. The example treatment device 100 includes a main unit 302 which preferably includes one or more processors 304 electrically coupled by an address/data bus 306 to one or more memory devices 308, other circuitry 310, and one or more interface circuits 312. The processor 304 may be any suitable processor or microcontroller. The memory 308 preferably includes volatile memory and non-volatile memory. Preferably, the memory 308 stores a software program that interacts with the other devices in the treatment device 100 as described below. This program may be executed by the processor 304 in any suitable manner.

The interface circuit 312 may be implemented using any suitable interface standard, such as an Ethernet interface and/or a Universal Serial Bus (USB) interface. One or more input devices 314, such as the start button 110, may be connected to the interface circuit 312 for starting the treatment device 100 and/or entering data and commands into the main unit 302. For example, the input device 314 may include a keyboard, mouse, touch screen, and/or any other suitable input device.

In addition, a card reader 316 may be connected to the interface circuit 312. The card reader 316 may be a smart card reader, a debit card reader, a thumb drive reader, and/or any other suitable memory reading device. The card reader 316 preferably accepts a card 318 or other memory device, such as a smart card, a debit card, and/or a thumb drive. In an embodiment, the card 318 is loaded with a value, and the treatment device 100 does not operate unless a predetermined value amount is detected on the card 318 by the card reader 316. Preferably, the value on the card 318 is reduced by the processor 304 via the card reader 316 each time the treatment device 100 is used. For example, a card 318 may initially store a value indicative one hundred ten minute illumination sessions. After the treatment device 100 is used once, the value on the card is reduced to indicate ninety nine remaining sessions.

When the value on the card 318 is reduced to a predetermined level, the treatment device 100 may indicate that the card level is low. For example, one or more displays, printers, speakers, alerts and/or other output devices 320 may be connected to the main unit 302 via the interface circuit 312, and when the card 318 is indicative of less than ten remaining sessions, the output device 320 may beep, the status LEDs 112 may flash, and/or other outputs may be generated. If a display is included, the display 216 may be a cathode ray tube (CRTs), liquid crystal displays (LCDs), a plasma device, or any other type of display. The display preferably generates visual displays of data generated during operation of the treatment device 100. For example, a display may be used to display a numerical value indicative of the time remaining in a session and/or the number of sessions (or minutes) remaining on a card 318. In addition, visual displays and/or other outputs may be used to prompt for human input, indicate other run time values, calculated values, data, etc. When the value stored by a card 318 is low or reduced to zero, the card 318 may be replenished or replaced as described in detail below.

In one embodiment, a card reader 316 with a valid card 318 is not required until a card reader 316 is attached. In this manner, the treatment device 100 may be sold outright as a stand alone unit or quickly modified to be a pay-per use unit. In addition, by placing a special card in the card reader 316, a treatment device 100 that is in the pay-per-use mode may be reverted to stand alone operation.

During a session, a plurality of treatment LEDs 322 illuminate in a preprogrammed manner. For example, the treatment device 100 may include a combination of visible red, visible amber, and non-visible infrared LEDs that may be modulated at a frequency of 73 Hertz and 584 Hertz in a 80%/20% weighted application mix. In addition, the treatment device may include a demonstration mode that continuously lights the treatment LEDs 322 at a low power level while the status LEDs 112 blink in a unique pattern.

One or more storage devices 324 may also be connected to the main unit 302 via the interface circuit 312. For example, a hard drive, CD drive, DVD drive, a flash device, and/or other storage devices may be connected to the main unit 302. The storage devices 318 may store any suitable type of data. The treatment device 100 may also exchange data with other devices 328 via a wireless transceiver and/or a connection to a network 326. The network connection may be any suitable type of network connection, such as an Ethernet connection, digital subscriber line (DSL), telephone line, coaxial cable, etc. The network 326 may be the Internet and/or some other data network, including, but not limited to, any suitable wide area network or local area network. It will be appreciated that any of the devices described in the examples herein may be directly connected to each other instead of over a network. In addition, any combination of devices described in the examples herein may be embodied in a single device.

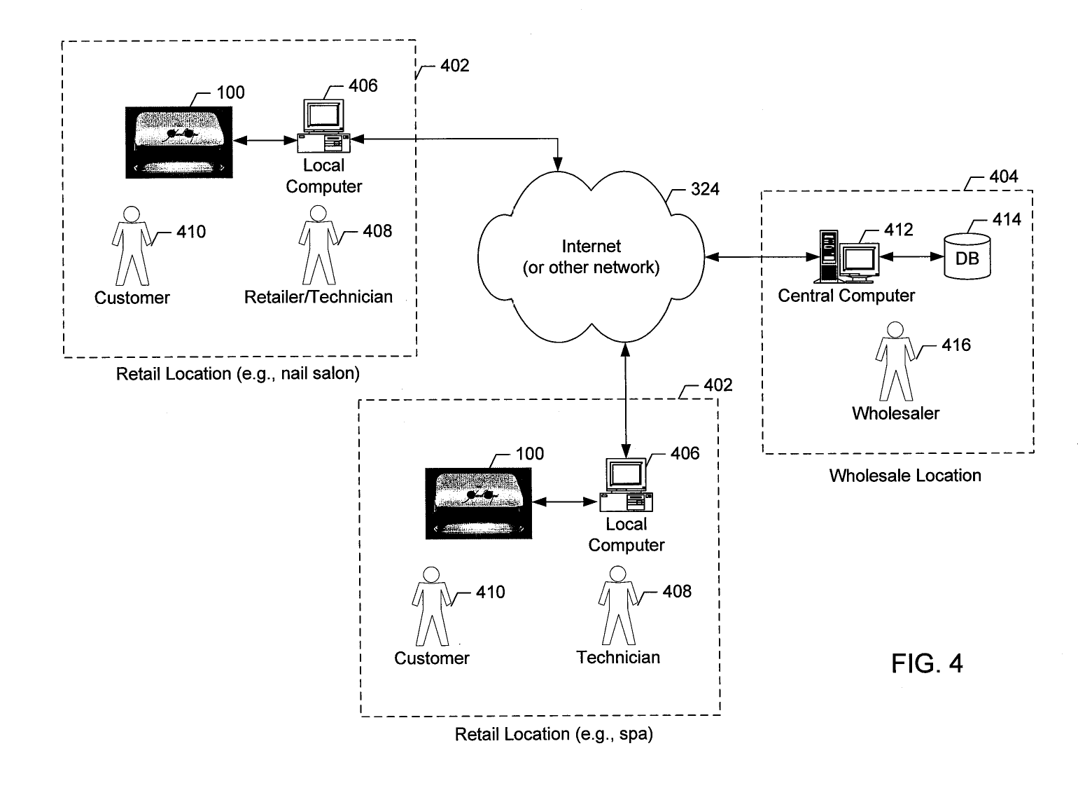

In one embodiment, cards 318 may be replenished via a connection to a network. A block diagram of an example network environment suitable for replenishing payment cards 318 is illustrated in FIG. 4. In this example, each treatment device 100 is connected to the network 324 via a local computer 406 or other suitable communication device at a retail location 402. Alternatively, the treatment device 100 may be connected directly to the network 324 via one or more interface circuits 326 as shown in FIG. 3. After one or more customers 410 use the treatment device 100, and the card 318 is partially or completely depleted of value, a technician 408 initiates a replenishing sequence programmed in the local computer 406 and/or the treatment device 100.

Once the replenishing sequence begins, the local computer 406 and/or the treatment device 100 preferably communicate with a central computer 412 at a wholesale location via the network 324. The central computer 412 may query a local and/or remote database 414 to determine if the particular treatment device 100 is authorized for replenishment. For example, each treatment device 100 may include a unique identification code. The unique identification code may be stored in the treatment device's internal memory 308, the card reader 316, the card 318, and/or the storage device 324. The central computer 412 may query the database 414 to determine if the retailer 408 associated with the unique identification code has sufficient credit to proceed with a card replenishment. For example, a wholesaler 416 may enter this credit information, in association with the unique identification code, upon receiving payment from the retailer 408. Alternatively, the wholesaler 416 may bill the retailer 408 after one or more card replenishments.

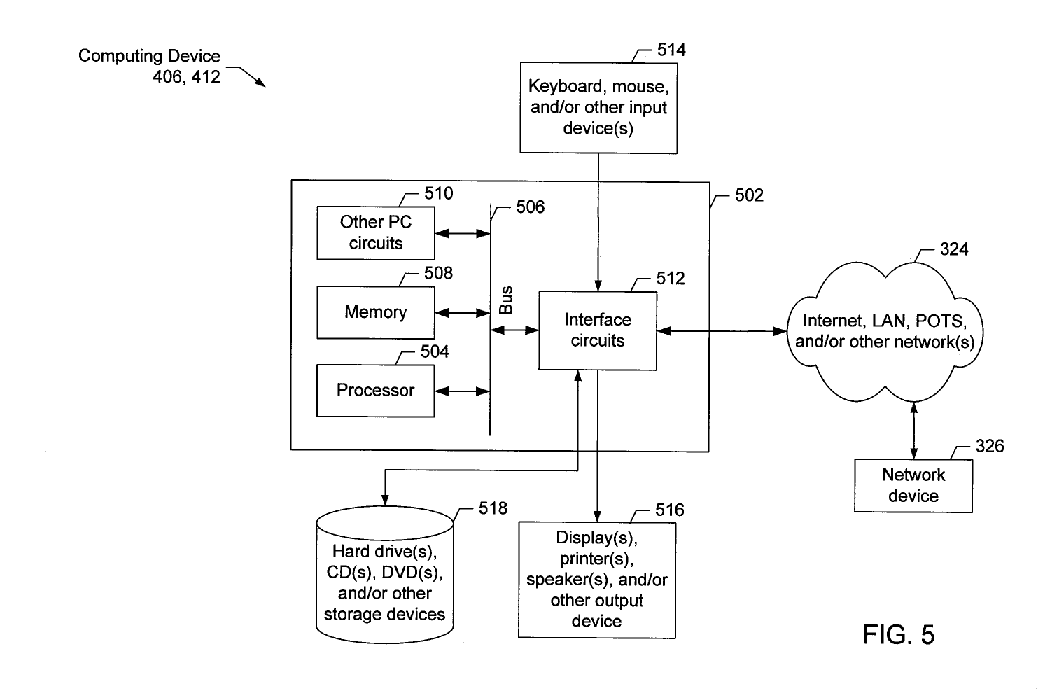

A block diagram of the electrical systems of an example computing device 406, 412 is illustrated in FIG. 5. Although the electrical systems of different computing devices 406, 412 may be similar, the structural differences between these devices are well known. For example, a typical handheld device is small and lightweight compared to a typical personal computer.

The example computing device 406, 412 includes a main unit 502 which preferably includes one or more processors 504 electrically coupled by an address/data bus 506 to one or more memory devices 508, other computer circuitry 510, and one or more interface circuits 512. The processor 504 may be any suitable processor, such as a microprocessor from the INTEL PENTIUM® family of microprocessors. The memory 508 preferably includes volatile memory and non-volatile memory. Preferably, the memory 508 stores a software program that interacts with the other devices in the system as described below. This program may be executed by the processor 504 in any suitable manner.

The interface circuit 512 may be implemented using any suitable interface standard, such as an Ethernet interface and/or a Universal Serial Bus (USB) interface. One or more input devices 514 may be connected to the interface circuit 512 for entering data and commands into the main unit 502. For example, the input device 514 may be a keyboard, mouse, touch screen, track pad, track ball, isopoint, and/or a voice recognition system.

One or more displays, printers, speakers, and/or other output devices 516 may also be connected to the main unit 502 via the interface circuit 512. The display 516 may be a cathode ray tube (CRTs), liquid crystal displays (LCDs), a plasma device, or any other type of display. The display 516 generates visual displays of data generated during operation of the computing device 406, 412. The visual displays may include prompts for human input, run time statistics, calculated values, data, etc.

One or more storage devices 518 may also be connected to the main unit 502 via the interface circuit 512. For example, a hard drive, CD drive, DVD drive, a flash device, and/or other storage devices may be connected to the main unit 502. The storage devices 518 may store any suitable type of data. The computing device 406, 412 may also exchange data with other devices 326 via a wireless transceiver and/or a connection to the network 324. The network connection may be any suitable type of network connection, such as an Ethernet connection, digital subscriber line (DSL), telephone line, coaxial cable, etc.

Users of the system may be required to register with the central computer 412. In such an instance, each user may choose a user identifier (e.g., e-mail address) and a password which may be required for the activation of services. The user identifier and password may be passed across the network 324 using encryption. Alternatively, the user identifier and/or password may be assigned by the central computer 412.

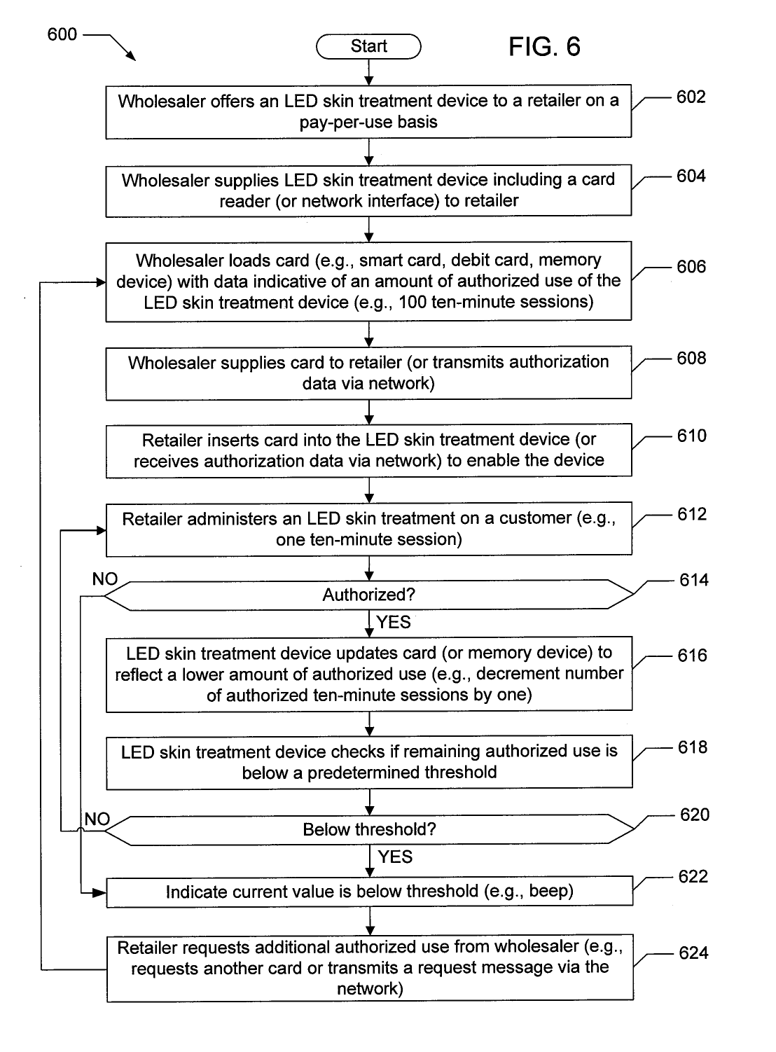

A flowchart of an example process 600 for paying for a skin treatment device on a per use basis is illustrated in FIG. 6. Preferably, the process 600 is embodied in one or more software programs which is stored in one or more memories and executed by one or more processors. For example, the process 600 may be software running on the treatment device 100, the card reader 316, the local computer 406 and/or the central computer 412. Although the process 600 is described with reference to the flowchart illustrated in FIG. 6, it will be appreciated that many other methods of performing the acts associated with the process 600 may be used. For example, the order of many of the steps may be changed, and some of the steps described may be optional.

Generally, a wholesaler supplies an LED skin treatment device 100 to a retailer 408. The retailer 408 pays the wholesaler on a per use basis, which is regulated by a digital key, such as a smart card attached to the treatment device 100 via a smart card reader. When the value of the digital key drops below a threshold (e.g., only ten treatment sessions remaining), an alert is generated by the treatment device 100. When the value of the digital key is reduced to zero, the treatment device 100 does not operate until the digital key is replenished or replaced.

More specifically, a wholesaler offers an LED skin treatment device 100 to a retailer 408 on a pay-per-use basis (block 602). For example, the wholesaler may offer to supply one or more LED skin treatment devices 100 to a retailer 408 at no initial charge. Instead, the retailer 408 agrees to pay the wholesaler $1.00 for each minute of treatment device use. In this manner, there is very little risk taken by the retailer 408. If the retailer 408 is able to sell skin treatments (e.g., at $2.00 per minute), the retailer 408 will likely do so at a profit. If the retailer 408 is not able to sell a significant number of skin treatments at some marked up rate, there is no finical lost to the retailer 408.

Preferably, the wholesaler supplies the treatment device 100 to the retailer 408 with a card reader 316 (block 604). The card reader 316 may be integrated into the treatment device 100, or the card reader 316 may be removably attached to the treatment device 100. For example, the card reader 316 may be removably attached to the treatment device 100 via a cable connection to a port on the back of the treatment device 100. The card reader 316 may be any suitable memory reading device. For example, the card reader may be a smart card reader, a debit card reader, a Universal Serial Bus (USB) receiver, etc.

Alternatively, or in addition, the wholesaler may supply the treatment device 100 to the retailer 408 with a network interface 312 (block 604). The network interface 312 may communicate directly with a network 326, or the network interface 312 may communicate indirectly with the network 326 via a local computer 406. For example, the treatment device 100 may be connected directly to the Internet and/or the telephone network via an internal or external modem. Alternatively, the treatment device 100 may be connected to the local computer 406 (e.g., via a serial cable), and the local computer 406 may be connect to the Internet in a well known manner.

When the retailer 408 is supplied with a treatment device 100 configured to work in conjunction with a card reader 316, the wholesaler 416 stores data on one or more cards 318 indicative of an amount of authorized treatment device use (block 606). For example, a code indicative of a number of purchased minutes and/or a number of purchased sessions may be stored on the card 318. Preferably, the wholesaler 416 uses a specially configured treatment device 100 and/or a central computer 412 to store the authorization data on the card 318. In one embodiment, the card 318 is a smart card including a microprocessor and/or a memory device that communicates with the treatment device 100 via the card reader 316. In another embodiment, the card 318 is a debit card with a magnetic strip that is read and can be modified by the card reader 316. In yet another embodiment, the card 318 is a flash memory device that can be read from and written to by the treatment device 100 via the card reader 316. In still another embodiment, the card 318 is a printed card with a machine readable symbol such as a bar code. In such an instance, the card reader 316 is preferably an optical sensing device such as a bar code scanner.

In any event, the wholesaler 416 supplies the card 318, with the data indicative of an authorized usage amount, to the retailer 408 (block 608). For example, the wholesaler 416 may mail the card 318 to the retailer 408, as part of initial treatment device shipment, periodically and/or in response to a request from the retailer 408. Alternatively, the wholesale location 404 may communicate with the retail location 402 electronically via the network 324 in order to replenish a card 318 and/or update a memory associated with the treatment device 100.

When the retailer 408 inserts a card 318 with sufficient value into the card reader 316, or the treatment device 100 receives authorization data from the network 324 (block 610), the treatment device 100 is enabled for use. The retailer 408 may then administer a treatment session on a customer (block 612). For example, a technician 408 may replace a clear plastic disposable liner in the bottom of the treatment device 100 and ask a customer to insert one or both of his/her hands into the treatment device 100. The technician 408 may then press the start button 110 to initiate a preprogrammed session (e.g., ten minutes).

If the treatment device 100 is authorized (block 614), the treatment session begins. As described above, status LEDs 112 then count down the time in the session in 25% increments. If the treatment device 100 is not authorized (block 614), the treatment session does not begin, and the treatment device 100 produces an error signal. For example, the treatment device 100 may produce a long beep via the alert transducer 320, and/or the treatment device 100 may indicate the error via the status LEDs 112.

In response to an authorized session being administered, the treatment device 100 communicates with the card 318 and/or a memory device (e.g., memory 308 and/or storage device 324) to indicate the use of a session (block 616). For example, an encrypted code may be modified to decrease the remaining amount of authorized sessions or minutes.

Each time the remaining authorization amount is decreased, the treatment device 100 checks if the remaining authorized time is below a threshold (block 618). For example, the treatment device 100 may check if the remaining authorized time is below ten sessions (e.g., one hundred minutes). If the remaining authorized time is not below the threshold (block 620), the retailer may administer additional sessions (block 612). If the remaining authorized time is below the threshold (block 620), the treatment device 100 indicates this condition (block 622). For example, the treatment device 100 may sound an alert and/or indicate the current low threshold condition on the status LEDs 112.

At any time, such as when the card value falls below the threshold or is completely depleted, the retailer may request additional minutes or sessions (block 624). The request from the retailer 408 may be manual (e.g., an e-mail message or a telephone call), or the request form the retailer 408 may be automated. For example, the treatment device 100 may connect to the network 324 to send a message to the central computer 412 when the treatment device's card value falls below a certain value.

In response to a replenishment request from the retailer 408, the wholesaler 416 may physically supply a new card 318 (e.g., physically via a mail system) or replenish an existing card 318 already at the retail location 402 (e.g., electronically via the network 324). Alternatively, the treatment device memory 308 may be updated by the central computer 412 automatically and/or in response to a request from the retailer 408. In such an instance, no card 318 is needed.

In summary, persons of ordinary skill in the art will readily appreciate that methods and apparatus for providing a skin treatment device on a pay-per-use basis have been provided. The foregoing description has been presented for the purposes of illustration and description. It is not intended to be exhaustive or to limit the invention to the exemplary embodiments disclosed. Many modifications and variations are possible in light of the above teachings. It is intended that the scope of the invention not be limited by this detailed description of examples.

What is claimed is:

1. A method of paying for a light emitting skin treatment device on a per use basis, the method comprising:
delivering the light emitting skin treatment device to a retailer, the light emitting skin treatment device requiring a digital key to be operated, the light emitting skin treatment device being programmed to modify data on the digital key to indicate use of the light emitting skin treatment device; and
delivering the digital key to the retailer, the digital key storing data indicative of a first duration of authorized use for the skin treatment device.

2. he method of claim 1, wherein the digital key is at least one of a smart card, a debit card, and a flash memory device.

3. The method of claim 1, wherein the data indicative of the first duration of authorized use is indicative of a number of authorized minutes.

4. The method of claim 1, wherein the data indicative of the first duration of authorized use is indicative of a number of authorized sessions.

5. The method of claim 1, wherein the light emitting skin treatment device is structured to accommodate two human hands simultaneously.

6. The method of claim 5, wherein the light emitting skin treatment device is structured to illuminate both sides of both hands simultaneously.

7. The method of claim 1, wherein the light emitting skin treatment device requires the digital key to be operated only after a digital key reader is attached to the light emitting skin treatment device.

8. The method of claim 1, including receiving the digital key from the retailer while the digital key stores data indicative of a second duration of authorized use for the skin treatment device, the second duration being shorter than the first duration.

9. The method of claim 8, wherein the second duration is zero.

10. The method of claim 8, including delivering a second different digital key to the retailer while the second digital key stores data indicative of the first duration of authorized use for the skin treatment device.

11. The method of claim 10, wherein the second digital key is delivered to the retailer prior to receiving the digital key from the retailer.

12. A method of operating a light emitting skin treatment device, the method comprising:
receiving a start signal;
determining if a memory device associated with the light emitting skin treatment device stores a first value indicative of a first amount of authorized use of the light emitting skin treatment device;
illuminating a plurality of light sources if the memory device stores the first value;
storing a second value on the memory device, the second value being indicative of a second amount of authorized use of the light emitting skin treatment device, the second amount of authorized use being less than the first amount of authorized use.

13. The method of claim 12, wherein the first value indicative of the first amount of authorized use is indicative of a number of authorized minutes.

14. The method of claim 12, wherein the first value indicative of the first amount of authorized use is indicative of a number of authorized sessions.

15. The method of claim 12, wherein the light emitting skin treatment device is structured to accommodate two human hands simultaneously.

16. The method of claim 15, wherein the light emitting skin treatment device is structured to illuminate both sides of both hands simultaneously.

17. The method of claim 12, wherein the light emitting skin treatment device requires the memory device to be operated only after a memory device reader is attached to the light emitting skin treatment device.

18. A pay per use skin treatment apparatus, the apparatus comprising:
a processor;
a memory device operatively coupled to the processor;
a user input device operatively coupled to the processor; and
a plurality of treatment LEDs operatively coupled to the processor; wherein the processor executes a software program to cause the processor to:
detect an activation of the user input device;
check the memory device for first data indicative of a first amount of authorized device usage time;
illuminate the plurality of treatment LEDs if the first data indicative of the first amount of authorized device usage time is above a threshold; and
store second data indicative of a second amount of authorized device usage time in the memory device, the second amount of authorized device usage time being less than the first amount of authorized device usage time.

19. The apparatus of claim 18, wherein the memory device includes a smart card.

20. The apparatus of claim 18, wherein the memory device includes a debit card.

21. The apparatus of claim 18, wherein the memory device includes a flash memory.

22. The apparatus of claim 18, wherein the memory device is attached to the skin treatment apparatus via an external reader.

23. The apparatus of claim 18, including a display device operatively coupled to the processor, wherein the display device indicates the first amount of authorized device usage time and the second amount of authorized device usage time.

24. The apparatus of claim 18, wherein the first amount of authorized device usage time is a number of authorized minutes.

25. The apparatus of claim 18, wherein the first amount of authorized device usage time is a number of authorized sessions.

26. The apparatus of claim 18, wherein the skin treatment apparatus is a light emitting skin treatment apparatus.

27. The apparatus of claim 18, wherein the skin treatment apparatus is a light emitting skin treatment apparatus that is structured to accommodate two human hands simultaneously.

28. The apparatus of claim 27, wherein the light emitting skin treatment apparatus is structured to illuminate both sides of both hands simultaneously.

29. The apparatus of claim 18, wherein the skin treatment apparatus requires the memory device to hold the first data to be operated only after the memory device is operatively coupled to the skin treatment device.

30. A pay per use skin treatment apparatus, the apparatus comprising:
a processor;
a memory device operatively coupled to the processor;
a network device operatively coupled to the processor;
a user input device operatively coupled to the processor; and
a plurality of treatment LEDs operatively coupled to the processor; wherein the processor executes a software program to cause the processor to:
detect an activation of the user input device;
check the memory device for first data indicative of a first amount of authorized device usage time, wherein the first data is received via the network device;
illuminate the plurality of treatment LEDs if the first data indicative of the first amount of authorized device usage time is above a threshold; and
store second data indicative of a second amount of authorized device usage time in the memory device, the second amount of authorized device usage time being less than the first amount of authorized device usage time.

31. The apparatus of claim 30, wherein the skin treatment apparatus is a light emitting skin treatment apparatus that is structured to accommodate two human hands simultaneously.

32. The apparatus of claim 31, wherein the light emitting skin treatment apparatus is structured to illuminate both sides of both hands simultaneously.

33. The apparatus of claim 30, wherein the skin treatment apparatus requires the memory device to hold the first data to be operated only after the memory device is operatively coupled to the skin treatment device.
